(19) United States
(12) Patent Application Publication
(10) Pub. No.: US 2021/0338721 A1
(43) Pub. Date: Nov. 4, 2021

(54) BIOAVAILABLE MINERALS FOR THE MITIGATION OF PATHOGENS IN ANIMALS, A MANUFACTURING METHOD AND A TREATMENT METHOD

(71) Applicant: Paul R. Simpson, Granite Falls, WA (US)

(72) Inventor: Paul R. Simpson, Granite Falls, WA (US)

(21) Appl. No.: 17/237,477

(22) Filed: Apr. 22, 2021

(30) Foreign Application Priority Data
May 4, 2020 (AU) 2020901404
Mar. 15, 2021 (AU) 2021201625

(51) Int. Cl. A61K 33/34; A61K 33/30; A61K 33/32; A61K 33/04; A61K 33/24; A61K 33/06; A61K 45/06; A61K 47/52; A61K 47/64

(52) U.S. Cl. CPC A61K 33/34; A61K 33/30; A61K 33/32; A61K 33/04; A61K 47/64; A61K 33/06; A61K 45/06; A61K 47/52; A61K 33/24

(57) ABSTRACT

The present invention relates to Animals with mineral deficiencies become viable to pathogen attack. An improved method of treating mineral deficiencies is therefore desired. This invention is employing bioavailable minerals in a liquid complex that are pharmaceutically acceptable carrier including water. The composition for use in the method may include other supporting animal nutrients such as botanical extracts, animal hormones, vitamins, and mineral supplements necessary for animal health.

### FIELD OF THE INVENTION

The present invention relates to a composition which includes bioavailable minerals for the mitigation of pathogens in creatures, especially in animals, a manufacturing method of the composition, and an improved method of treating mineral deficiencies.

### BACKGROUND OF THE INVENTION

Animals have mechanisms to contain and or eliminate attacks from pathogens. They utilize a variety of methods to inhibit the development of pathogens. Insects, parasites, fungus, viruses, bacteria are subject to a variety of chemical changes that an animal may utilize to discourage the pathogens from replicating and leading to their consequential demise.

An early example is Man's use of salt to preserve food, the principle in the chemical composition is sodium, a metal.

An animal with adequate mineral availability will adjust the transport mechanisms within the animal to mitigate attacks from pathogens. These are accomplished with REDOX (reduction-oxidation) and measured by the balance of pH & energy within the animal. FIG. 4 illustrates REDOX the relationship of Electrical Charge (Eh) to Acid-Alkalinity (pH). To generate energy, an animal requires minerals. Two dissimilar minerals in an electrolyte generate 1.5 volts and when cells operate in series, higher voltages. Through electrolysis an animal can separate minerals from compounds sufficient to maintain their energy requirements and an ideal REDOX of Eh4.5-pH6.50. This varies with each animal and the conditions where it grows.

Other mechanisms active within an animal are SOD (Superoxide Dismutase) and ROS (Reactive Oxygen Species) which require energy from mineral participation.

Creatures, especially animals, can suffer from mineral deficiencies that result in undesirable states, including attacks from pathogens caused by insects, parasites, fungus, viruses, and bacteria. A variety of methods are known for treating these undesirable states, but none have been entirely satisfactory. An improved method for treating mineral deficiencies is therefore desired. The discussion above is merely provided for general background information and not intended to be used as an aid in determining the scope of the claimed subject matters.

Studies have shown that many skin abnormalities are caused by the presence of non-typical or mutated cells. By eliminating non-typical or mutated anaerobic cells and latent toxicity, growth of new, healthy, aerobic cells is facilitated.

In one aspect of the invention there is proposed a composition for treating mineral deficiencies, comprising:
at least one complex cation and inorganic coordination complexes formed by the coordinate bond formation between an electropositive mineral cation and molecular groups;
the inorganic coordination complexes including a protective cation mineral ligand carrier, that specifically releases the mineral cation to supply areas that require such a mineral;
wherein the complex cation and inorganic coordination complexes is absorbed by diffusion and the mineral cation can then move freely throughout the internal animal or human circulation, without the cation binding to targets that do not have mineral deficiency for this cation.

In preference the ligand cation complex is ammonia or super-oxide dismutase.

In preference the ligand cation complex is ammonium hydrogen sulphate ((NH₄)HSO₄).

In preference the complex cation is mineral cation.

In preference the mineral cation is zinc or/and copper cation.

In preference the composition further comprises one of manganese, magnesium, cobalt, chromium, molybdenum, selenium, and vanadium.

In preference the composition further comprises one of botanical extracts, animal hormones, vitamins, and mineral supplements necessary for animal health.

In preference the ratio of zinc to copper of the composition is 7:2.

In preference the composition further comprises the composition has a pH of less than 1.0.

In a further aspect of the invention there is proposed a method of manufacturing a composition for treating mineral deficiencies of claim 1, comprising:
preparing a solution of ammonium hydrogen sulphate ((NH4)HSO₄) using sulfuric acid (H₂SO₄); diluting the ammonium hydrogen sulphate with water to form a mixture;
adding sulfuric acid;
adding a solution iron sulphate (FeSO4) and water; and/or
adding a solution manganese sulphate (MgSO4) and water; and/or
adding a solution zinc sulphate (ZnSO4) and water; and/or
adding a solution copper sulphate (CuSO4) and water; and
agitating the mixture until the ammonium hydrogen sulphate, the sulfuric acid, the water, the manganese sulphate, the iron sulphate, the copper sulphate and the zinc sulphate are blended together to form a zinc metal-ligand complex comprising a first plurality of ammonia ligands and a copper metal-ligand complex comprising a second plurality of ammonia ligands.

In preference the method further comprises;
adding at least one delivery agent to the composition prior to the step of treating.

In preference the method of treating mineral deficiencies comprises treating a creature with a composition, the composition being formed by the above.

In preference the creature is an animal.

### BRIEF DESCRIPTION OF THE DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of the Invention in any way. The Detailed Description will refer to several drawings as follows.

FIG. 1 is a drawing of a typical cell structure;

FIG. 2 is a drawing of the KREBS CYCLE of cells;

FIG. 3 is a drawing of the body requirement for minerals for conversion of Super Oxide Dismutase (SOD);

FIG. 4 is a table depicting REDOX the relationship of Electrical Charge (Eh) to Acid-Alkalinity (pH);

FIG. 5 is the production flow chart of the composition according to an embodiment of the present invention;

FIGS. 6A, 6B, 6C are pictures of the treatment effect of using the product made from a composition of the present invention;

FIG. 7 is a picture of the wound area of the dog before applying the product with an insert of after applying the product;

FIG. 8 is a picture of the wound area of the hours after applying the product with an insert of before applying the product;

FIGS. 9A, 9B, 9C are pictures of treatment effect of using the product made from a composition of the present invention;

FIGS. 10A, 10B, 10C are pictures of treatment effect of using the product made from a composition of the present invention;

FIGS. 11A, 11B are pictures of treatment effect of using the product made from a composition of the present invention;

FIGS. 12A, 12B are pictures of treatment effect of using the product made from a composition of the present invention;

### BRIEF DESCRIPTION OF THE INVENTION

The following detailed description of the invention refers to the accompanying drawings. Wherever possible, the same reference numbers will be used throughout the drawings and the following description to refer to the same and like parts. Dimensions of certain parts shown in the drawings may have been modified and/or exaggerated for the purposes of clarity or illustration.

This invention proposes employing bioavailable minerals in a liquid complex that are pharmaceutically acceptable carriers including water which is used for treating mineral deficiencies in animals, such as spray, mists, injection, rinse or oral. Moreover, the bioavailable minerals can be included in formula drugs or even in the form of gels, agars or dermal or transdermal patches. Consequently, a composition including a cation and a cation ligand carrier is formed which can increase productivity and reduce disease severity and insect damage in animals.

Element Zinc is essential to healthy immune function especially in the various skin layers, recognized as a possible treatment for cancers, powerful anti-inflammatory and shown to disrupt viral activity both inside and outside the cell. Copper utilized in the mitochondrial membrane generates most of the energy needed to power biochemical reactions. High concentration levels of Copper and Zinc are toxic to abnormal cells causing normal, programmed cell death. Zinc, Copper and Silver Dihydrogen Citrate are toxic to pathogenic microbes.

Transition metals manganese (Mn), iron (Fe), cobalt (Co), nickel (Ni), copper (Cu), and zinc (Zn) found in the D-block of the periodic table are present in biological systems as metal complexes. These ions are able to form coordinate covalent bonds with species called ligands. These ligands have atoms that have electron pairs that are available to be shared with metallic ions that have a positive charge and empty coordination sphere orbitals. Most organic molecules and ions that have oxygen and nitrogen will have these paired electrons available for bonding with metal ions. When a metal ion forms a coordinate bond with a ligand, a complex is formed. When this complex has a net charge, it is a complex ion. Neutral complexes will not be soluble in the aqueous environment of cells. Depending on the charge of the metal ion and ligand, both positive and negative complexes can be formed, and will be more soluble in biological environs.

There is an equilibrium established in an organism when a complex is present. Depending on the strength of the bond between the ligand and metal ion, a small amount of the free metal ion will be present. This equilibrium is pH dependent and also dependent on other ligands that may be present in the organism.

The efficiency of the composition of the present invention is directly proportional to the amount of free metal ions, for instance zinc ions, copper ions or their sum. Sub nano-molar concentrations of zinc would exist as an aqua-complex, i.e., complexed by water molecules. In some embodiments, the metal ions of the compositions are zinc which is complexed with amine through the dermis where it is picked up by zinc transport proteins and other naturally occurring ligands and transported to the cell. Then, topical application of zinc will lead to a localized higher concentration of free zinc and enhance absorption into the surrounding tissue which therefore treats diseases because of mineral deficiencies.

An embodiment of the present invention provides topical products composed of a proprietary mineral complex formulated to regenerate healthy cellular function. This unique mineral complex which contains ionic copper-zinc enhances new cell growth with a nutrient rich combination of vitamins that synergistically control and eliminate viral, bacterial, and fungal infections. First and foremost, the principles of the biology working mechanism of the compositions or products provided by the present invention and the reasons why it can treat, improve, or cure pathogens and regenerate cell in animal or human bodies are going to be discussed.

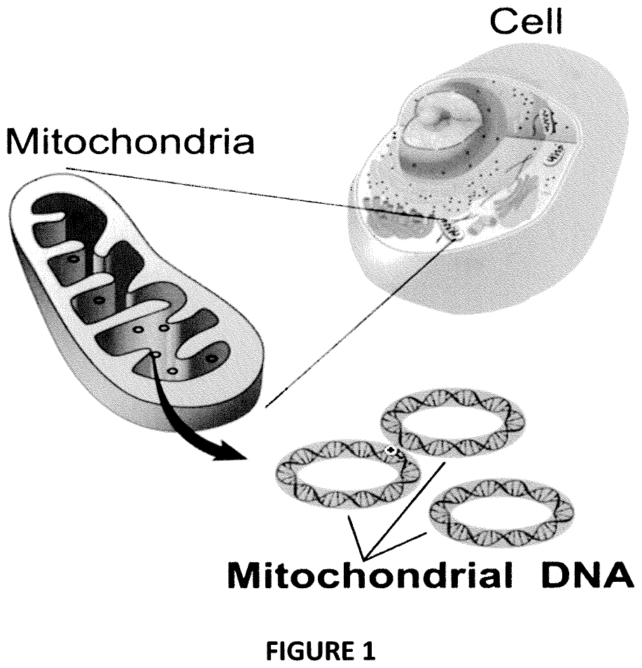

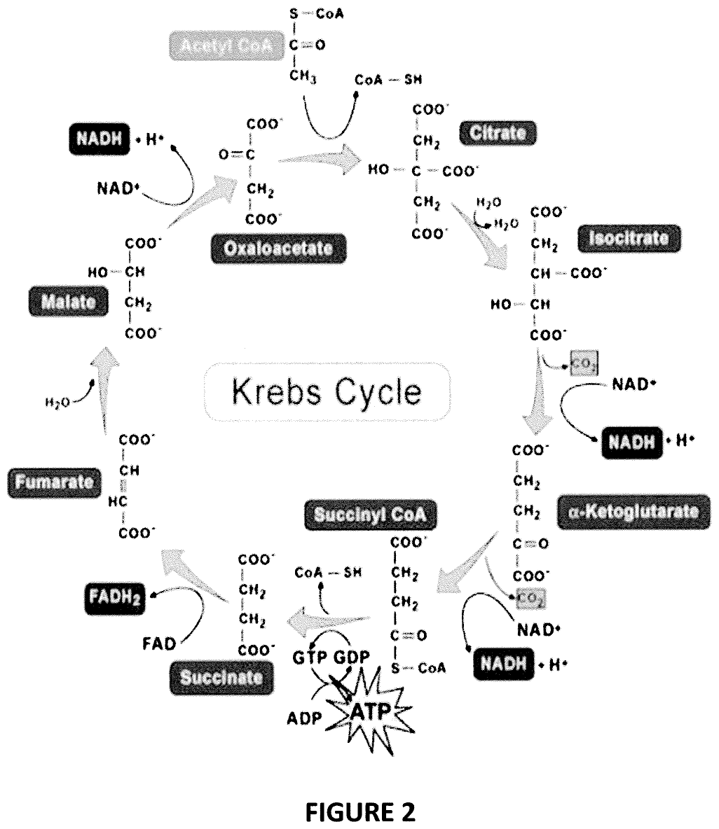

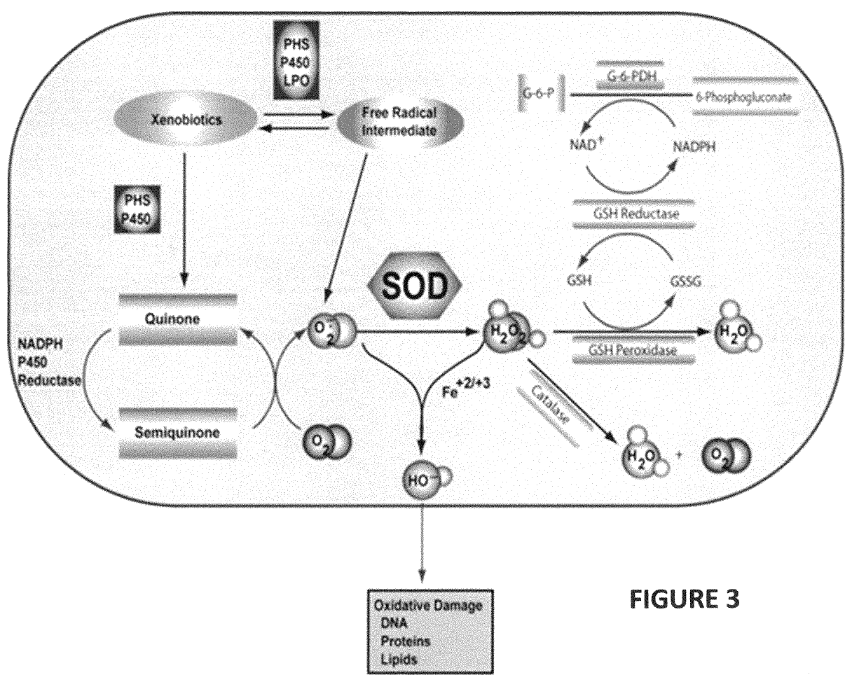

On one hand, healthy cells, as shown in FIG. 1, require energy to survive. Carbs, fats and proteins are eventually broken down into two carbon units (Acetyl-CoA) passing through the Krebs Cycle as shown in FIG. 2 to become electrons tied up in NADH and FADH₂ and available for pumping electrons into the cell inter-membrane, and to be further transformed to the inter-membrane space into the matrix couple, in ADP into ATP, to give the cell body energy. On the other hand, enzymes NADPH Oxidase breaks down oxygen into a Superoxide as shown in FIG. 3. A buildup of Superoxide causes loss of energy and cell death. SOD (Superoxide Dismutase) such as that found in the present invention formula, converts Superoxide's to Oxygen (O2) and Water (H2O). To function properly, the preferred SOD requires the mineral ions of Zinc and Copper (Zn, Cu Superoxide Dismutase). Accordingly, the mode of action of the compositions of the present invention is designed to treat several illnesses by targeting mutated cells and promoting healthy cell growth. Further small alterations of the product consisting of the compositions provided by the present invention can be purposed to target a variety of localized pathologies.

The present invented formula contains ionic Zinc and Copper as a Zn, Cu Superoxide Dismutase and may also contain ionic Manganese and ionic Iron for additional effectiveness.

The present invention uses ionic mineral complexes that are capable of penetrating through and being readily absorbed into deep dermal layers of the skin of animals or humans providing an efficient pathway for delivery of vital nutrients to tissue beneath the skin, while having little adverse effect on normal cell operations. Further, ionic mineral complexes are capable of penetrating cell membranes at a rapid pace through simple diffusion.

Following, the formula of a composition for treating mineral deficiencies in creatures, especially in animals, provided by the present invention will be described in detail. The composition includes at least one complex cation and inorganic coordination complexes formed by the coordinate bond formation between an electropositive mineral cation (positive) and molecular groups that have unshared electron pairs. The inorganic coordination complexes include a protective cation mineral ligand carrier that specifically releases the mineral to supply areas of the animal that require such a mineral.

The ligand cation complex is absorbed by diffusion into an animal and can then move freely throughout the internal animal circulation, without the cation binding to targets that do not have mineral deficiency for this cation. The ligand cation complex of the composition is ammonia or super-oxide dismutase. In some embodiment, the ligand cation complex is ammonium hydrogen sulphate ((NH₄)HSO₄). The complex cation of the composition is mineral cation. In some embodiment, the mineral cation is zinc cation or copper cation or zinc and copper cations.

In preference the composition further includes at least one of manganese, magnesium, cobalt, chromium, molybdenum, selenium, and vanadium.

In preference the composition further includes botanical extracts, animal hormones, vitamins, and mineral supplements necessary for animal health.

The ammonia ligand configuration also acts as an artificial super-oxide dismutase (SOD) which acts as a free oxygen scavenger to alleviate animal stress, for example, under drought and mechanical damage.

Testimonials have reported beneficial results in the treatment of insects and worms. These results indicate an extended deterrence against various animal issues with no detrimental side effects. These preliminary results suggest that an animal with balanced nutrition particularly zinc, copper and sulphur is able to create its own resistances and deterrents. The detailed descriptions for the testimonials will be presented accompany with the treatment effect pictures at last of this specification.

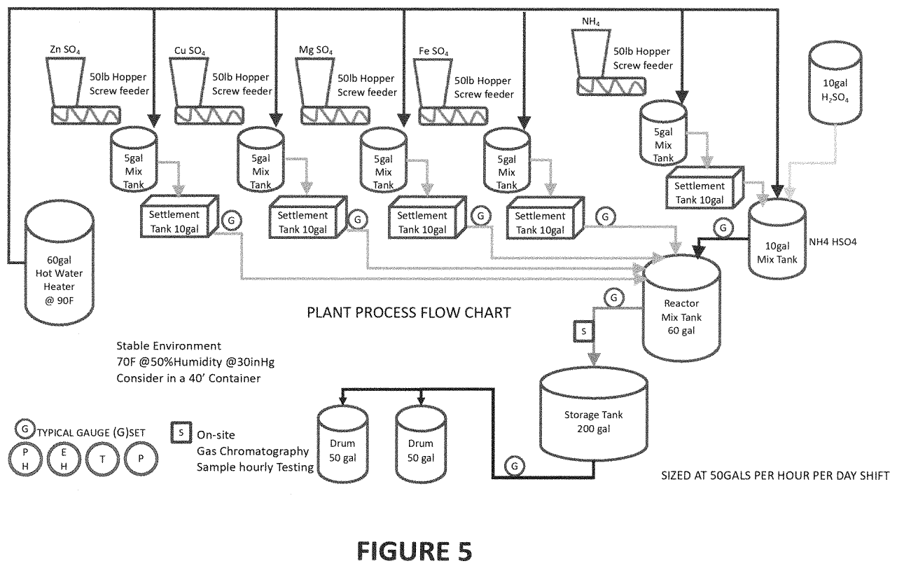

The flow chart in FIG. 5 illustrates the method of production where elements are combined to manufacture the formulation provided by the present invention. This method reduces the heat generated from exothermic reactions when combining these elements.

A method of supplementing nutritional intake of a living animal, the method comprises treating an animal with a composition, the composition being formed by steps of:
a. preparing a solution of ammonium hydrogen sulphate ((NH₄)HSO₄) using sulfuric acid (H₂SO₄);
b. diluting the ammonium hydrogen sulphate with water to form a mixture;
c. adding sulfuric acid;
d. adding a solution iron sulphate (FeSO₄) and water;
e. adding a solution manganese sulphate (MgSO₄) and water;
f. adding a solution zinc sulphate (ZnSO₄) and water;
g. adding a solution copper sulphate (CuSO₄) and water;
h. agitating the mixture until the ammonium hydrogen sulphate, the sulfuric acid, the water, the manganese sulphate, the iron sulphate, the copper sulphate and the zinc sulphate are blended together to form a zinc metal-ligand complex comprising a first plurality of ammonia ligands and a copper metal-ligand complex comprising a second plurality of ammonia ligands and a manganese metal-ligand complex comprising a third plurality of ammonia ligands and a iron metal-ligand complex comprising a fourth plurality of ammonia ligands.

Consequently, the mixture forms the composition.

In a preferred embodiment, beneficial, symbiotic, supplemental ingredients are added to the minerals/acids complex mixture which would increase the efficacy of the final compound. The product is in the form of cream that contains the following ingredients: Deionized Water (Aqua), Mineral Oil, Propylene Glycol, Isopropyl Palmitate, Vegetable Glycerin, Stearyl Alcohol, Cetyl Alcohol, Dimethicone, Steareth-21, Steareth-2, PEG-50 Shea Butter, Vitis vinifera (Grape) Seed Extract, Co-Q10 Enzyme (Ubiquinone), Tocopheryl Acetate (Vitamin E), Niacinamide (Vitamin B3), Sodium Ascorbyl Phosphate (Vitamin C), Glutathione, Panthenol (Pro-Vitamin B5), Pyridoxin (Vitamin B6), Copper Sulfate (and) Zinc Sulfate (and) Sulfuric Acid, Sodium Hydroxide, Silver Dihydrogen Citrate (and) Citric Acid, Xanthan Gum, Magnesium Aluminum Silicate, Tetrasodium EDTA, Citric Acid, Saccharomyces Lysate Extract, Phospholipids, Lecithin, Sodium Milkamidopropyl PG-Diammonium Chloride Phosphate, Methylparaben, Propylparaben, Diazolidinyl Urea.

In another preferred embodiment, the product is in the form of liquid, which includes the following ingredients: Purified Water (Aqua), Paraffinum Liquidum, Propylene Glycol, Isopropyl Palmitate, Vegetable Glycerin, Stearyl Alcohol, Cetyl Alcohol, Steareth-21, Steareth-2, Tetrahydrodiferuloylmethane (Tetrahydrocurcuminoids), Aloe Barbadensis (Aloe vera) Leaf Juice Extract, Copper Sulfate (and) Zinc Sulfate (and) Ammonium Sulfate (and) Sulfuric Acid, Sodium Hydroxide, Silver Dihydrogen Citrate (and) Citric Acid, Tetrahexyldecyl Ascorbate (Vitamin C), Grapeseed Extract (Vitis vinifera), Co-Q10 Enzyme (Ubiquinone), Tocopheryl Acetate (Vitamin E Acetate), Arnica montana (Arnica Flower) Extract, Glutathione, Panthenol (Pro-Vitamin B5), Pyridoxin (Vitamin B6), Superoxidase Dismutase, Xanthan Gum, Magnesium Aluminum Silicate, Tetrasodium EDTA, Citric Acid, Saccharomyces Lysate Extract, Phospholipids, Lecithin, Methyparaben, Propylparaben, Diazolidinyl Urea.

In some preferred embodiments, the compositions include a high level of sulfur in the form of sulfate which functions as an active secondary transport complex. This sulfate has a negative charge and is not solvated or wrapped up in transport proteins or ligands like positive zinc and copper ions. Movement of sulfate into the cell is dependent on a number of factors including concentrations of other ions, pH and concentration gradients across the membrane. If influx of sulfate is favored it can aid in transport of copper and zinc ions in their movement into the cell. This is especially useful when the active ATP transport process is inhibited. This sulfur complex can operate synergistically with the mineral complex by accelerating absorption of affected cells. The high level of free sulfur can be transported to various locations and may speed reconstitution of damaged tissue affected by disease.

Sulfur is a non-metallic acidic micro-mineral usually consumed as part of a larger zinc, copper compound whose expression is not usually considered an aid to mitigation of aberrant diseased cells. However, the benefits of sulfur are well known and a formulation that combines a high-sulfur content (NH₄HSO₄) base with minerals that contain sulfur (zinc sulfate, etc.) provides an abundance of free sulfur that may accumulate in those regions of the human body requiring attention.

The preferred mineral would be a sulfate for that reason but is not necessary for success of free sulfur. Benefits of sulfur include boosting the immune system and providing pain relief to targeted cells. The mechanism by which free sulfur produced in this mixture operates similarly to the operative mode of glucosamine sulfate, chondroitin sulfate, and dimethyl sulfoxide.

In preference, the method of manufacturing the composition further comprising: adding at least one delivery agent to the composition prior to the step of treating. The delivery agent selected from the group consisting of a surfactant, dimethyl sulfoxide, a urea-based compound, a detergent, a hygroscopic compound, and combinations thereof.

In preference the ratio of zinc to copper of the composition is 7:2.

In preference the composition has a pH of less than 1.0.

In preference the method of manufacturing the composition further comprising a step of analyzing the animal to determine a specific nutrient required and, wherein the composition further includes a nutrient matched to the specific nutrient required.

In preference the method of manufacturing the composition further comprising adding at least one of manganese, magnesium, cobalt, chromium, molybdenum, selenium, and vanadium into the composition.

In preference the method of manufacturing the composition further comprising adding at least one of an animal hormone, an auxin, or an animal extract into the composition.

In a further form of the invention there proposed a method of supplementing bio-mineral nutritional intake of a healthy, living animal, the method comprising steps of treating a healthy animal with a composition, the composition being formed by:
i. preparing a solution of ammonium hydrogen sulphate ((NH₄)HSO₄) using sulfuric acid (H₂SO₄);
j. diluting the ammonium hydrogen sulphate with water to for a mixture;
k. adding solutions of manganese sulphate and water, iron sulphate and water, zinc sulphate (ZnSO₄) and water, copper sulphate (CuSO₄) and water to the mixture, wherein the ratio of zinc to copper to manganese to iron is 7:2:1:1;
l. agitating the mixture until the ammonium hydrogen sulphate, the sulfuric acid, the water, the solution of copper sulphate and the solution of zinc sulphate are blended together to form a zinc metal-ligand complex comprising a first plurality of ammonia ligands and a copper metal-ligand complex comprising a second plurality of ammonia ligands;
m. further dilution of the mixture with water may be required to form the composition, wherein the composition has a pH of less than 1.0.

The present invention also discloses the composition is applied for treating the healthy animal comprises a topical application (cream or spray), injection, irrigation or oral.

The step of treating the healthy animal comprises adding the composition to a topical application to the affected area of the animal. Upon introduction the animal, the composition bonds with a super oxide dismutase composition and is transported to mineral-deficient parts of the animal.

The product formulation as manufactured provides a delivery system for moving mineral ions to the mineral deficient areas in animals using highly bioavailable cations through a complex ligand system. Ammonia ligands form a bond with the free metallic ions, especially zinc and copper to regions of the animal that require such minerals. Other minerals to be named follow the same pattern of dispersal throughout the animal in a systemic nature. The animal's natural metabolism will discard excess metals as described in the metabolic pathway's descriptions although an over-whelming amount of the composition is possible and may cause necrosis in the animal. The ionic nature of the formulation allows easy access to areas of the animal with deficiencies and provides a formulation that is stress relieving. Systemic delivery of targeted, and minerals create more robust animals. Benefits to animals accomplished organically, without the use of additive or subtractive genetic modify-techniques and is therefore considered non-GMO. Significantly smaller quantities of additive minerals and growth compounds compared to conventional husbandry. Organic formula and dilute application requirements should substantially reduce economic, social, ecological, and regulatory concerns associated with runoff into water sources. Use of less water compared to conventional farming methods. The ability to deploy self-contained animal growth systems for use in hostile environments such as the polar regions, deserts. The disclosure further provides a method of reducing the time to maturity comprising supplementing nutritional intake in a living animal by the disclosed method. The disclosure further provides a method of increasing farm yields for food comprising supplementing nutritional intake in a living animal by the disclosed method. The disclosure further provides a method of creating more robust animals by systemic delivery superior to current technologies.

The present disclosure relates to a method of supplementing nutritional intake in a living animal, the method comprising steps of: treating an animal with a composition, the composition being formed by: adding ingredients of water, H₂ SO₄, (NH₄) of targeted nutrients and minerals to crops comprising supplementing nutritional intake of a living animal by the disclosed method. The disclosure further provides a method of increasing the nutritional quality of animal derived produce, including the SO₄, copper and zinc into a mixture; agitating the mixture until the ingredients are blended together; diluting the mixture with water to form the composition.

The present disclosure relates to a method of supplementing nutritional intake of a healthy, living animal, the method comprising steps of: treating a healthy animal with a composition, the composition being formed by: adding ingredients of water, H₂SO₄, (NH₄)₂SO₄, copper and zinc into a mixture; agitating the mixture until the ingredients are blended together; diluting the mixture with water.

It has been surprisingly found that the disclosed methods, which utilize a bioavailable mineral composition comprising water, H₂SO₄, (NH₄)₂SO₄, copper and zinc leads to many positive effects in animals, for example healthy animals, and in particular in food crop animals. Animals treated according to the disclosed method have been found to be faster growing, healthier and/or more robust. Additionally, the disclosed method may allow for fewer applications of the treatment compared to other known mineral treatments leading to considerable cost and time savings. The benefits of the disclosed methods for supplementing nutritional intake in living animals may be summarized as including one or more of improvements:
Faster time to maturity, with higher farm yields
Improved fertility
Increased biomass of animals
Systemic delivery of targeted nutrients and minerals to animals to create more robust animals
Ability to increase the nutritional quality of animal derived products, (butter, meat, eggs),
Benefits to animals accomplished organically, without the use of additive or subtractive genetic modification techniques and is therefore considered non-GMO
Significantly smaller quantities of additive minerals and growth compounds compared to conventional farming
Organic formula and dilute application requirements should substantially reduce economic, social, ecological, and regulatory concerns associated with runoff into water sources
Use of less water compared to conventional farming methods
The ability to deploy self-contained animal growth systems for use in hostile environments such as the polar regions, deserts, underwater habitats, and other extreme locations.

The disclosure further provides a method of reducing the time to maturity comprising supplementing nutritional intake of a living animal by the disclosed method.

The disclosure further provides a method of increasing farm yields for food comprising supplementing nutritional intake of a living animal by the disclosed method.

The disclosure further provides a method of creating more robust animals by systemic delivery of targeted nutrients and minerals comprising supplementing nutritional intake of a living animal by the disclosed method

The disclosure further provides a method of increasing the nutritional quality of animal products, including the potential to engineer nutrition into our food, comprising supplementing nutritional intake of a living animal by the disclosed method.

The disclosure further provides a method of using less water compared to conventional farming methods comprising supplementing nutritional intake of a living animal by the disclosed method.

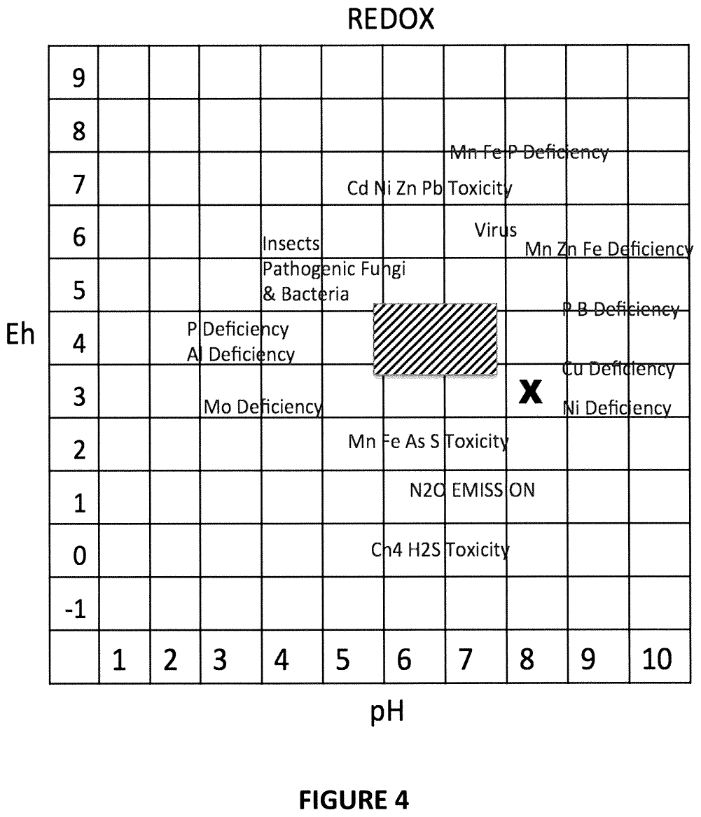

As shown in FIG. 4, the relationship of Electrical Charge (Eh) to Acid-Alkalinity (pH) of REDOX. The cross-hatch shaded area is the general sweet spot for optimal animal development. This varies with specific animals. Also, pathogens have a preferred sweet spot. By using the patented solution, we adjust the redox in an animal to the sweet spot whereby the animal is able to mitigate pathogens.

The manufacturing process described below produces a complex having ammonia ligand bonds with specific cations. The cations are carried by the ligand bonds and protected from being immediately bonded with the first available negative ions thus enabling free movement between and within the cells of the animal. Each of the minerals will be processed in the acid/base solution resulting in products that have a high acidity value, yet not being corrosive to living tissue. A noticeable concentration of reactive ammonia is also produced by the acid/base reaction. Complex cations and inorganic coordination complexes are formed that can move the cations in a relatively stable fashion and allow transport throughout the animal.

A solution containing a mix of the prepared mineral(s) may contain only one of the minerals and additional supplements or all the mentioned minerals plus selected supplements to achieve the desired effect. For example, a zinc deficiency may only require zinc and a small amount of copper to balance (Mineral Inter-relationships) the effect of the possibility of too much zinc and other supplements (urea for example) to reinforce the effect of the mineral. All the minerals will act independent of one another much as gases do in Dalton's Law and be assimilated by the animal on an as-needed basis. For example, copper and zinc can be antagonistic to one another in an animal, but the two minerals can be put into the formulation at the right proportions and counter act the influence of one against the other. In some embodiments of the present invention, the copper and zinc also act to counterbalanced each other physiologically on a basis of 7:2 mole ratio (zinc to copper) and with the ammonia (NH3) form the composition the subject of this invention, a form of ligand complex. The ligand travels through the selective membrane (epidermis) and travels through the animal's xylem and/or phloem to a mineral deficient tissue where a physiological change will occur, and the minerals are un-encapsulated from the ligand complex and are usable as part of the metabolic pathway in the animal.

The ligand formation is obtained during manufacture. However, the use of the ionic mineral in the formulation can be utilized to form an artificial superoxide dismutase (SOD). Animals normally form SODs however since silicon is necessary in most animals and is to support the structure. An example of incorporation of the composition as described with reference to the superoxide dismutase (SOD) cycle will use copper (Cu) and zinc (Zn). The composition will be incorporated into the SOD on an as needed basis and attached to the mineral complex to make a Cu-SOD, a Zn-SOD, or a Cu/Zn-SOD. The enzyme superoxide dismutase catalyzes dismutation of super oxide into oxygen and hydrogen peroxide. Therefore, it is an important antioxidant defense in almost all cells exposed to oxygen. The SOD catalyzed dismutation of super oxide may be written using the following half reactions:

M(″+¹)+SOD+O²−M(″⋅)+SOD+O

M(+)+SOD+O²+2H+−M(″+¹)+SOD+H₂O₂

Where M may be, but is not limited to:
(a) Cu (n=l); (b) Zn (n=2); (c) Mn (n=2); Fe (n=2); (e) Ni (n=2).

In this reaction the oxidation state of the metal cation oscillates between n and n+l. Several common forms of SOD 55 exist and are proteins co-factored with copper (Cu) and zinc (Zn), manganese (Mn), iron (Fe) or nickel (Ni). Cytosols of almost all eukaryotic cells contain SODs and combine with copper and zinc (Cu—Zn-SOD)s. The Cu—Zn SOD and design is a homodimer of molecular weight of approximately 32,500. The Cu and Zn are joined primarily by hydrophobic and electrostatic interactions. The ligand complexes of copper and zinc are histidine side chains whereas the ligand complexes of manganese ions are three histidine side chains. The incorporation of the ligand complex into the SOD enables the ligand complex to travel throughout the animal in a protected form and without compromising the effectiveness of the ligand complex. Once the SOD with the ligand complex reaches a target cell the cat-ion within the ligand complex is released into the cell.

The resulting formulation can be prepared in many ways for application and will vary with the intended use. Formulations prepared for treatment for animal mineral deficiency will require selection of the proper formulation containing the necessary minerals. The formulations are stable at a pH near or below pH 1.0 in a wide variety of carriers. However, the active composition is prepared using the liquid. Additionally, the low pH of the composition may be diminished if the composition crystallizes.

The mineral cation ingredients in the active composition will vary in proportion depending on the intended use and be added in certain formulation depending on the type and purpose thereof. The added inert ingredients used in the formulation will also vary considerably depending on the site and purpose of the application. Other active compounds may be added if the proposed components prove to be beneficial to the formulation. However, the basic ingredients are known to be effective without any additional components.

Additional elements may be added to the composition that aid in the overall effectiveness of the formula. For example, animal extracts and urea have demonstrated additional effectiveness for certain animal groups. Auxins and animal hormones may be useful for specific uses.

Treatment of mineral deficiencies and/or promoting general health of the animal may be carried out by topical application. The composition can also be applied as an injectable in cases where there is a special effort to incorporate minerals into an animal to ensure absorption. The animal may be soaked, sprayed, drenched or submerged in a diluted solution (the ratio of the composition to the water is 1:2 and above). A newborn animal may be treated by spraying, misting, drenching directly. The formulation is systemic to the dermal surface of the skin.

The formulation can also be applied in a food source with the formulation being applied to the animal. The formulations can also be incorporated into creams, gels, sprays, mists, agars and dermal and transdermal patches.

The bioavailable minerals for the mitigation of pathogens in animals provided by the present invention has a significant and positive effect on reduction of pathogen attack by increasing the animal's responses by discouraging a pathogen's preference as a reproduction environment. It aids farmers with animal husbandry with early immune development in animal offspring. It may accelerate maturation rate of young animals. It also aids animals with recovery from pathogen attack. With optimal concentration, it can be customized to fit the critical need of the animal, balancing development with production of immunity and mitigating pathogen attack.

This invention reduces the level of Superoxide's in cells which when over produced, due to extreme stress may stimulate ROS (Reactive Oxygen Species) which kills healthy cells. By preventing the over production of the Superoxides and ROS, the invented formula can thereby stimulate healthy cells and prevent various viruses from reproducing.

Other benefits of the products manufactured by the method of the present invention include, highly absorbable, precisely targeted application to problem infection sites, abnormal skin growths or discolorations, efficiently delivery of concentrates of active ingredients to targeted skin tissue and promoting health skin renewal to all the skin layers.

Some testimonials prove the effects of the products made from the compositions of the present invention will be illustrated in the following.

The first testimonial is a horse with deep lacerations from the attack by a bear. It was chased into a barbed wire fence by the bear.

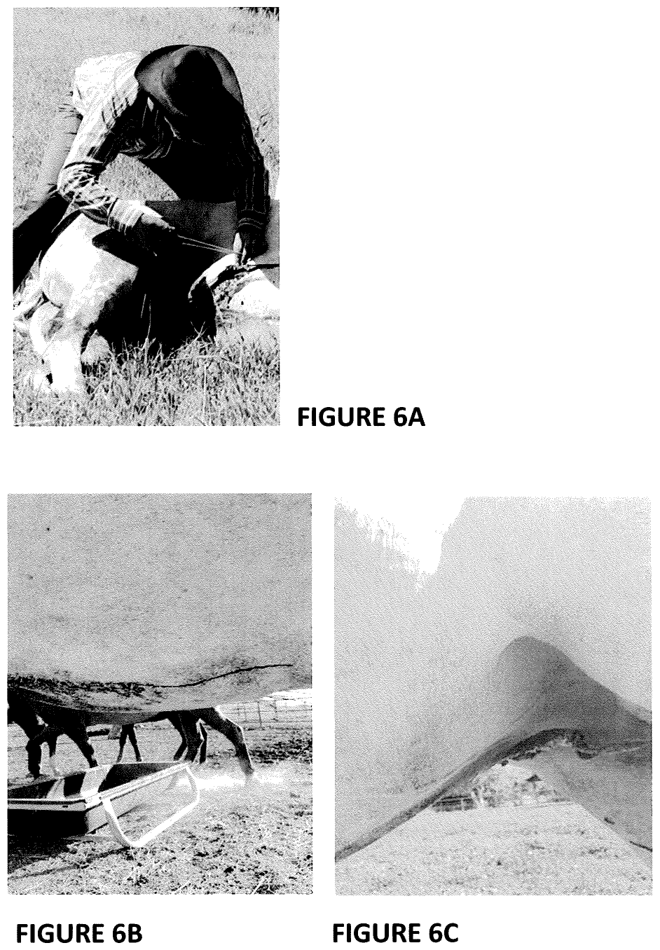

The Veterinarin anesthetized the damaged area and stitched the wound together as shown in FIG. 6A and applying 2 layers of the product which is made from the compositions of the present invention. After several sessions of applications, it had proven to be worthy for several reasons:
1. It was summer and the wound, not being covered, was constantly under attack by pests. The ABS kept pests away because the wound was odorless;
2. It was an easy application with spray bottle. The ingredients are made slightly to a gel consistency so it stays where it is sprayed;
3. The ingredients contain natural additions that target pain, dryness, and healing powers;
4. The hair cover returned to its natural color as shown in FIG. 6C.

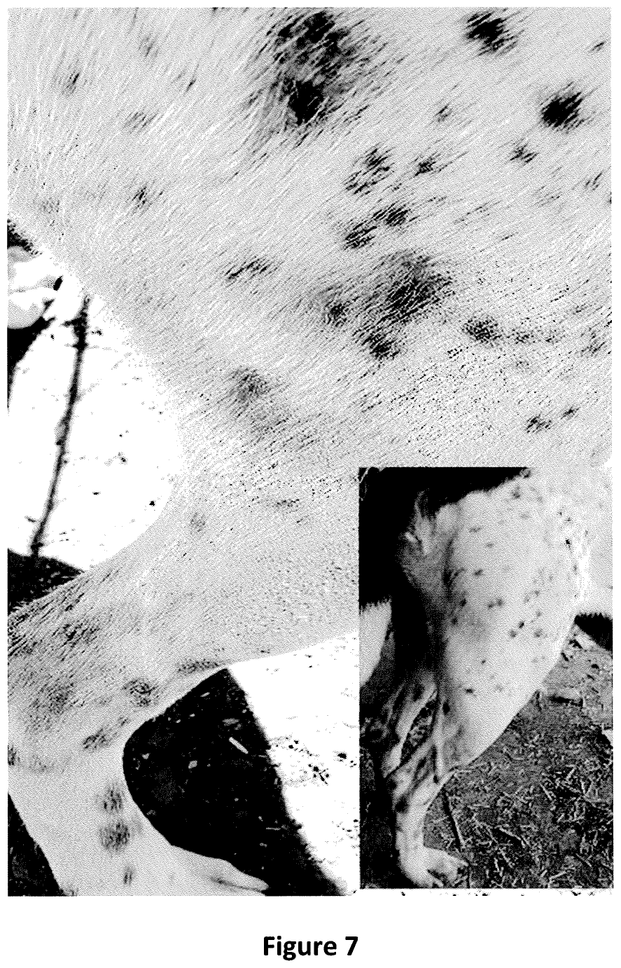

The second testimonial is a three years old dog with serious insect attacks. The product made from the compositions of the present invention is easily sprayed with formula which disappears into the wound area within seconds. It had been applied once a day for 9 days and stopped itching and healed within 2 weeks. The formula kept the pests off the wounds. Note the reoccurrence of hair growth to original color as shown in FIG. 7.

The third testimonial is a severe gall wound over a ten years old whither horse. Treatment without dressing in summer months should have been a challenge but was proven to be successful with the application of the formula comes from the present invention twice a day for 19 days.

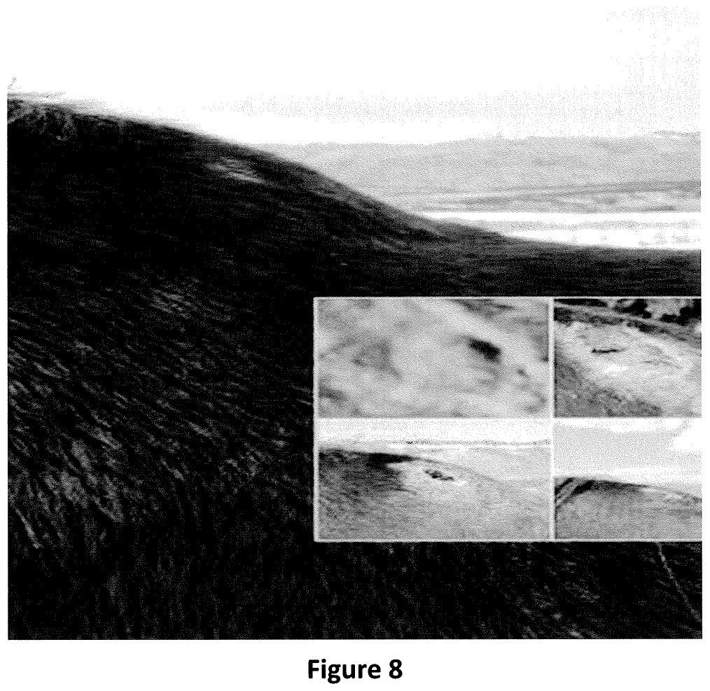

The unique ingredients proved to deter pests from the wound allowing the healing process to occur faster. Please note the wound area is in fact healing with full hair covering as shown in FIG. 8 which is an after application picture with an insert of before application.

The fourth testimonial is a sporting hound with a scrape from a bad fall. The product made from the composition of the present invention was applied twice daily for 6 days resulting in a healed wound. It should be noticed that after the application of this product flies and pests would not bother the wound.

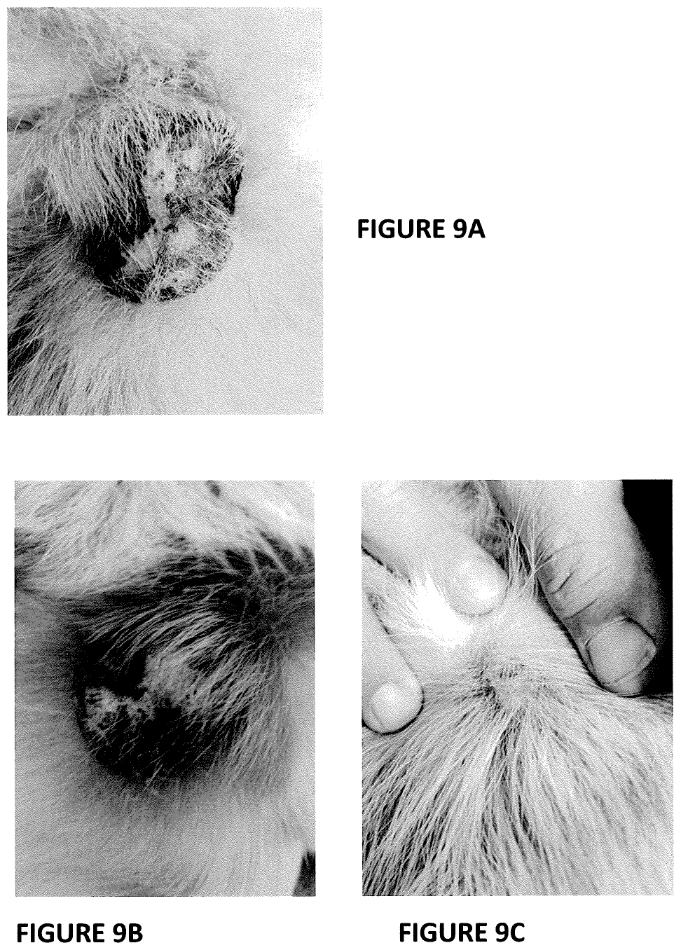

The fifth testimonial is an Aussie dog with possible bite as shown in FIG. 9A. The product made from the composition of the present invention was applied once daily for 8 days. The dog was very irritated and acted like the wound was itching. The product definitely was calming and the hair after healing came back to its natural color as shown in FIGS. 9B and 9C.

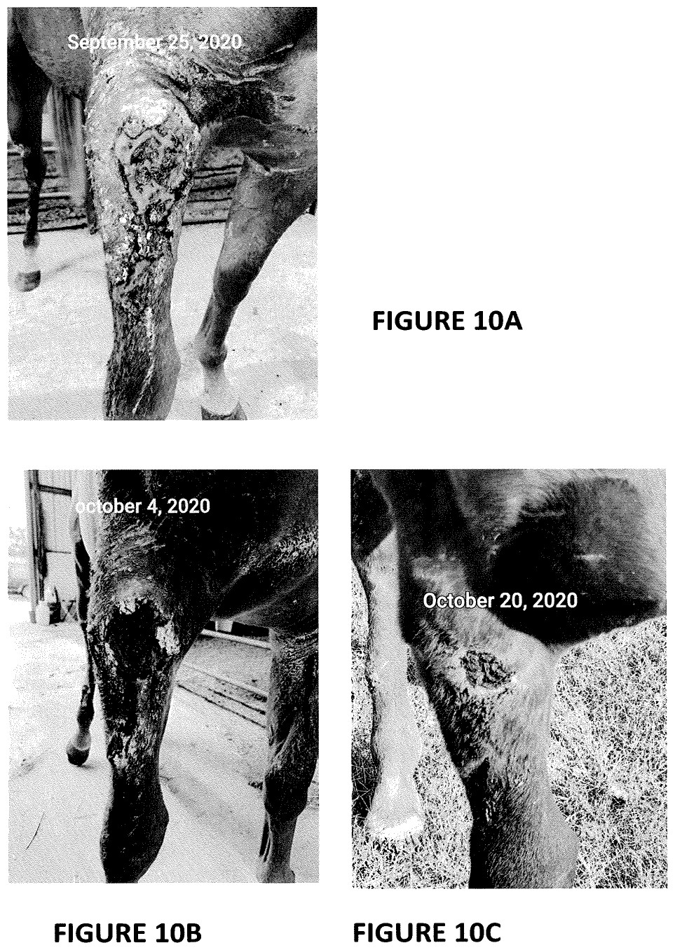

The sixth testimonial is a performance horse tangled in barbed wire. This horse is an expensive highly trained performance mare. It was found two days after the occurrence, tangled in a barbed wire fence. The Veterinarian suggested putting the mare down or a skin graph as the only resolutions, informed owner that the wound was too vast for stitching as shown in FIG. 10A. So, the wound could not being covered and was constantly under attack by pests. The product made from the composition of the present invention named ABS with Sun-FROST technology was applied and has proven to be worthy for the following reasons:
The treatment eliminates odor;
It was an easy application with spray bottle;
The ingredients are made slightly to a gel consistency, so it stays where it is sprayed;
The ingredients contain natural additions that target pain, dryness, and healing powers as shown in FIG. 10B;
The hair cover returned to its natural color as shown in FIG. 10C.

Generally, it has become unacceptable to evaluate technology on animals. Consequently, our studies have been conducted on consenting adults, and because of the non-invasive application potential side effects mitigated. Additionally, results are delivered without the need for speculation and interpretation. Testimonials have reported beneficial results in the topical application of the product namely Sundancer Topical. These results indicate an extended deterrence against various pathogens issues.

These preliminary results suggest that an animal with balanced nutrition particularly Zinc, Copper and Sulphur is able to create its own resistances and deterrents.

A woman testimoanial was born in Barbados 23 Oct. 1957. She supposed that the formula cream itself which contains the compositions of the present invention is not a cure but, it identifies mutated cells (caused by any virus) to your own body's immune system so that it can eliminate them while making healthy cells healthier. She started using the product namely Sundancer Technical as (Superior Skin Repair) cream properly about 6 months ago and the results are convincing. The weakest part of her body is chest. As soon as she gets sick which is not often goes straight to her chest. After using the cream for one week, what started to come from her chest was a deep green as she had a chest infection. She would normally be on antibiotics immediately but, instead she persisted with the cream and now what is coming from her chest is thick but colorless.

She can now breathe normally as the cream not only eliminated the infection which was suppressed for years with antibiotics. It also allows the body's immune system to permanently rid her chest of this chronic virus forever. What is also happening is the cream is removing so many dead skin cells and allowing new cells to be produced that the woman looks 15 to 20 years younger.

There are some side effects of this product; the main one is that some days you feel fatigued. It is natural to feel like this when one has over 60 years of viruses infesting within the body as the body's immune system fights them. It is recommended to use this product for between 6 months to a year depending on age.

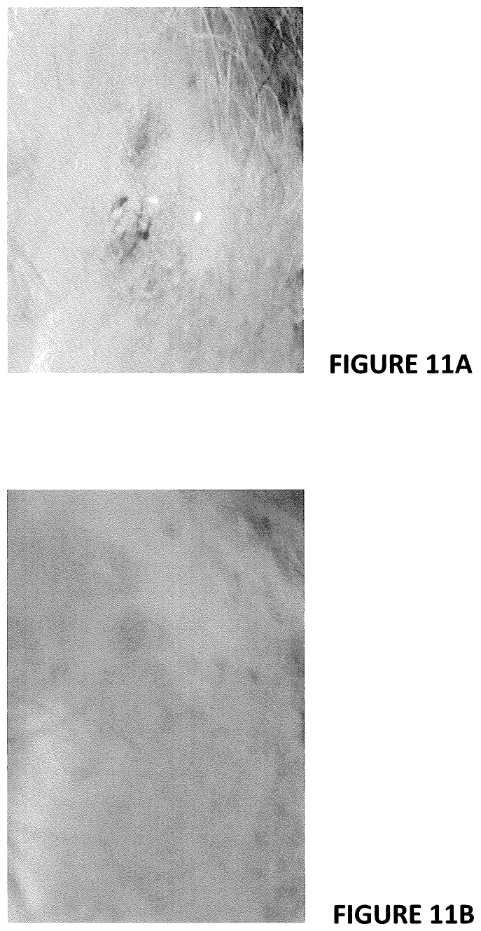

A 78-year-old man with an undiagnosed lesion below the left temple. During a routine medical visit the doctor noticed a small a suspicious mole approximately 1 inch from the subjects left eye. The growth continued to get larger over a period of 3+ month period. The man began using the Sundancer formula 3 days on side of face twice a day with Band-Aid before documenting with pictures. The recovery after two weeks and 5 weeks are shown in FIGS. 11A and 11B.

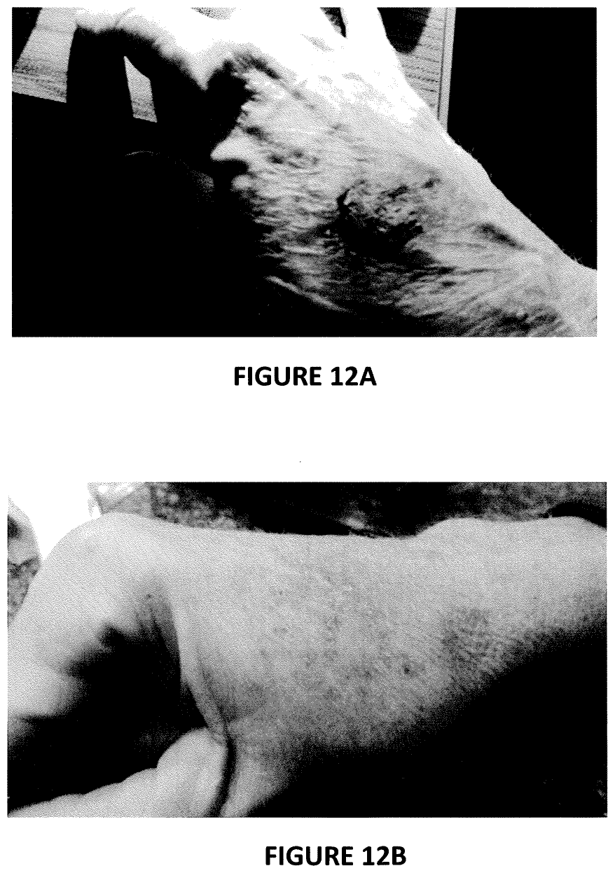

A male aged 72 on July, 2013 had a dog scratch on top of the right hand. Applying the Sundancer cream formula on large mass right hand started 2 days after incident) three times a day. The wound at the first day is shown in FIG. 12A and the recovery after the 18th days of the application of the product is shown in FIG. 12B.

A girl with cellulitis nose and right intranasal abscess had taken prescription of Bactrim and Sulfamethoxazole. Both twice a day and some Bactrim ointment to put inside and outside of the nose and got a shot with 2 antibiotics in it. Another prescription for codeine was given. And she was diagnosed positive for MRSA. But all the aforeaside drugs didn't seem to be working and the antibiotics caused dry rash all over the body of the patient. Then, the product contains the compositions of the present invention was applied 5-6 times a day on the patient's nose, face, temples, back of neck and under left armpit where there is a cyst the month before she got the MRSA. Around one week later than the first use of the product, any new growth of pathogens had been impeded Improvement had been observed every day after the patient started the product use. The body rash and fever were gone and the nose was less swollen and just pink now instead of bright red and bulbous.

A women had a disease called Amyotrophic Lateral Sclerosis (ALS) or some people may know it as Lou Gehrig's disease, an autoimmune disorder. This disease makes the loss of movements in the body, including speech and then even breathing. To date the patient has struggled more than 7 years with this disease. She began using the product from Apr. 13, 2013. Since then she has experienced many improvements on her abilities to move. She started noticing muscle movements on the shoulder/arm area also on the back, and she could move my legs and hands a lot more. On Jun. 16, 2013 her first Electromyographic scan showed that my energy assessment was 360. The energy assessment for a normal person is 10 and the lower the better. She continued using the Formula, and also kept noticing improvements. On Jul. 19, 2013 she got my second EMG scan and the new energy assessment number was 230.

1. A composition for treating mineral deficiencies, comprising:
at least one complex cation and inorganic coordination complexes formed by the coordinate bond formation between an electropositive mineral cation and molecular groups;
the inorganic coordination complexes including a protective cation mineral ligand carrier, that specifically releases the mineral cation to supply areas that require such a mineral;
wherein the complex cation and inorganic coordination complexes is absorbed by diffusion and the mineral cation can then move freely throughout the internal animal or human circulation, without the cation binding to targets that do not have mineral deficiency for this cation.

2. The composition of claim 1, wherein the ligand cation complex is ammonia or super-oxide dismutase.

3. The composition of claim 1, wherein the ligand cation complex is ammonium hydrogen sulphate ((NH₄)HSO₄).

4. The composition of claim 1, wherein the complex cation is mineral cation.

5. The composition of claim 4, wherein the mineral cation is zinc or/and copper cation.

6. The composition of claim 1, further comprising one of manganese, magnesium, cobalt, chromium, molybdenum, selenium, and vanadium.

7. The composition of claim 1, further comprising one of botanical extracts, animal hormones, vitamins, and mineral supplements necessary for animal health.

8. The composition of claim 1, wherein the ratio of zinc to copper of the composition is 7:2.

9. The composition of claim 1, wherein the composition has a pH of less than 1.0.

10. A method of manufacturing a composition for treating mineral deficiencies of claim 1, comprising:
preparing a solution of ammonium hydrogen sulphate ((NH4)HSO₄) using sulfuric acid (H₂SO₄);
diluting the ammonium hydrogen sulphate with water to form a mixture;
adding sulfuric acid;
adding a solution iron sulphate (FeSO4) and water; and/or
adding a solution manganese sulphate (MgSO4) and water; and/or
adding a solution zinc sulphate (ZnSO4) and water; and/or
adding a solution copper sulphate (CuSO4) and water; and
agitating the mixture until the ammonium hydrogen sulphate, the sulfuric acid, the water, the manganese sulphate, the iron sulphate, the copper sulphate and the zinc sulphate are blended together to form a zinc metal-ligand complex comprising a first plurality of ammonia ligands and a copper metal-ligand complex comprising a second plurality of ammonia ligands.

11. The method of claim 10, further comprising:
adding at least one delivery agent to the composition prior to the step of treating.

12. A method of treating mineral deficiencies comprising: treating a creature with a composition, the composition being formed by steps of claim 10.

13. The method of claim 12, wherein the creature is an animal.
